(19) United States
(12) Patent Application Publication
(10) Pub. No.: US 2009/0180971 A1
(43) Pub. Date: Jul. 16, 2009

(54) Method for the prevention and/or activation of defense mechanisms in the human body against conditions, disorders or diseases relating to the presence of free radicals, such as: environmental pollution, aging or smoking

(75) Inventor: German Alfredo Martinez Marcos, Cali (CO)

Correspondence Address: FURR LAW FIRM, 2622 DEBOLT ROAD, UTICA, OH, 43080, US

(73) Assignee: Pedro Luis Abeldt

(21) Appl. No.: 12/013,466

(22) Filed: Jan. 13, 2008

(51) Int. Cl. A61K 36/886; A61K 36/752; A61K 31/05; A61K 9/68

(52) U.S. Cl. 424/48; 424/736; 424/744; 514/731; 424/440

(57) ABSTRACT

The invention provides a method for the prevention and/or activation of defense mechanisms in the human body against conditions, disorders or diseases relating to the presence of free radicals, such as; environmental pollution, aging or smoking by rising the administration to a mammal, namely a human being, of an effective amount of a phytonutrient-and-citric-based edible dosage, during or after tobacco consumption and which is intended to prevent pathologies, conditions, disorders or diseases relating to the presence of free radicals in the human body. Likewise, the therapeutic method of the invention enables activation of defense mechanisms in the human body to reduce conditions, disorders or diseases relating to the presence of free radicals in the body.

### FIELD OF THE INVENTION

The invention relates to a method for the prevention and/or activation of defense mechanisms in the human body against conditions, disorders or diseases relating to the presence of free radicals, such as environmental pollution or smoking, which comprises the administration of an effective amount of an edible phytonutrient-and-citric-based dosage.

### BACKGROUND OF THE INVENTION

In big cities, air pollution results from the release to the atmosphere of exhaust fumes from internal combustion engines, household heating devices and industries either as gases, fumes or solid particles, that are capable of being held in suspension, in amounts that exceed the standards, thus harming the life and health of both human beings and animals as well as plants.

Polluted air affects our daily lives, producing diverse symptoms in our organism, such as eye irritation, conjunctive membrane disorders, and irritation of airways, genetic damage, hormone action alterations and cancer.

The tobacco compounds in cigarettes are a substantial source of chemical pollutants such as free radicals, carcinogenics and irritants. A direct correlation has been proven between smoking and an increased risk of developing lung and extrapulmonary cancer (pharynx, larynx, esophagus, tongue, etc), heart and lung diseases (chronic bronchitis and emphysema) as well as vascular disorders. Recently the WHO has estimated that there are 1.1 billion smokers and that 500 million of them will die of smoking-related diseases if they continue with this activity.

The smoke of tobacco is composed of two phases, gas and tar. Both phases contain high levels of oxidants, free radicals, nitrogen oxides and other potentially toxic chemicals (in a 200 mg cigarette smoke sample, 85% is nitrogen N₂, oxygen O₂ and carbon dioxide CO₂, having an 8% of wet particulate material over tar, the remaining 7% is carbon monoxide CO). The effects caused or triggered by tobacco products may be acute and chronic events and may even lead to death.

The cigarette's tar may lead to DNA strand breakage, whose effect is synergistically augmented by the presence of nitric acid (NO), which is formed in lungs from compounds such as catechol and 1,4-hydroquinone present in the tar (Yoshie, et al. Carcinogenesis 1997; 18:1359-1363). Likewise, tobacco has been linked to an increased risk of lung and bladder cancer and hepatocellular carcinoma (Hietanen, E et al. Environ. Health Perspectives. 1997; 105:749-754) and ulcer perforations (Svanes, C et al. Gut. 1997; 41:177-180).

The free radicals are linked as probable causes of cancer, coronary diseases and aging. These free radicals correspond to molecule fragments having increased reactivity as they are in a state of electronic imbalance—at least one unpaired electron. A free radical may destroy an enzyme, protein or merely the cell, however the damage is usually more widespread since the free radical usually triggers a sequence of free-radical-producing reactions that leads to the damage of numerous body components.

Five basic types of damage caused by free radicals that accelerate the aging process have been identified:
1. Lipid peroxidation, in which free radicals initiate the reaction by attacking fats causing rancidity thereof and producing more free radicals.
2. Protein and molecular DNA entanglement.
3. Damage to the cellular membrane by direct reaction with the components of the membrane, interfering with the absorption and waste elimination processes.
4. Damage to liposomes, caused by the reaction of free radicals before the liposomal membrane, producing free enzymes inside the cytoplasm that damage other cellular components.
5. Accumulation of lipofuscin, which interferes in the cellular chemistry.

Among the most harmful free radicals we find the peroxide radical (O₂), the hydroxyl radical (OH), the lipid peroxide radical (LOO) and the hydrogen peroxide (H₂O₂). The human body has enzymes whose function is to defend it from these agents, in this group we find the dismutase superoxide—which destroys the superoxide radical—, the catalase—Vitamin E producer—and glutathione peroxidase—which stops lipid peroxidation and reduces hydrogen peroxide to water.

DAMAGES CAUSED BY FREE RADICALS

| FREE RADICAL | DAMAGE |
|---|---|
| Superoxide | Erithrocyte Lysis |
| | Pulmonary damage |
| | Degradation of synovial fluid |
| Single Oxygen | Ocular damage |
| Hydroxile Radicals | Breakage of single and double |
| | DNA strands |
| | Peroxidation of cellular |
| | membranes |
| Nitrogen Dioxide | Lipid peroxidation |
| | Irreversible respiratory damage |
| Transition Metals | Initiation of lipid perodixidation |
| Chloroform Radicals | Lipid peroxidation, liver damage |
| Quinone Radicals | DNA, RNA damage |
| | Chemical carcinogenesis |
| | Lipid Peroxidation |
| Formation of Bipyridyl Radicals | Inhibition of fatty acids synthesis |
| | Cataracts |
| Phenyldiazine Radicals | Hemoglobin denaturation |
| | Lipid Peroxidation |

Furthermore, carbon monoxide is responsible for hemoglobin reduction due to the formation of metahemoglobin or carboxihemoglobin, a resulting product from hemoglobin oxidation. Studies indicate that nonsmokers living in industrialized countries lose around 1% of hemoglobin due to the above, while smokers lose over 15% depending on cigarette consumption and exposure time. This means that smokers have a higher risk of having vital organs not receiving an adequate oxygen supply, which may contribute to the high number of heart attacks among the smoking population.

The methods currently employed to preclude the harmful effects associated with tobacco consumption include:
The suspension and/or substitution of the habit of smoking for another habit.
Nicotine replacement by means of using transdermal patches and tablets: U.S. Pat. No. 4,832,994 describes the use of pills that contain silver acetate and sucrose; these pills have an acceptable taste upon dissolution in the absence of cigarette but produce an unpleasant taste in presence of tobacco smoke or tar. U.S. Pat. No. 3,876,804 and 4176890 teach chewing gums with different agents (silver ammonia and malthol and/or ethilmalthol compounds) that produce an unpleasant taste when the subject smokes. U.S. Pat. No. 4,920,989 discloses the combined use of transdermal patches and spray for delivering nicotine to the patient to the bloodstream and oral cavity, respectively. It is possible to find currently in the market the products Nicorette®, chewing gum and inhaler by Pharmacia & Upjohn, Nicotinell® chewing gum and patches from Ciba and Nicolan®, trasdermal patches from Astra.
The treatment with therapeutics agents as antagonists of nicotine receptors and antidepressants or ansiolitics (U.S. Pat. No. 6,197,827, U.S. Pat. No. 6,582,737, U.S. Pat. No. 6,495,605).
The usage of filters for removing potentially toxic agents contained in the smoke and the tar: U.S. Pat. No. 6,615,843 discloses a filter containing a composition made from minerals and micropulverized antioxidant agents that perform the function of chelation, coupling, diffusion and interception of gas, particles and free radicals of tobacco during the consumption process.

Likewise, numerous compositions have been reported in the literature that include vitamins, minerals and trace elements for the treatment of diseases derived from tobacco consumption (EP205133, WO9731546, DE19649730, DE4312071, FR2693876, CN1138428) as well as some composition such as liposomes that include non-enzymatic amphipathic antioxidants for the treatment of diseases caused by free radicals (U.S. Pat. No. 6,764,693).

Antioxidants are indispensable organic compounds that take part in numerous biochemical processes. Antioxidants have proven to be effective in the removal of free radicals and consequently, they have the capacity of preventing the carcinogenesis in the epithelial tissues of the bronchi, trachea, stomach, skin, uterus and prostate in humans and animals, effects that have been demonstrated both in vivo and in vitro. Several mechanisms have been suggested to explain these anticarcinogenic effects. Currently there are several ongoing studies to determine the relation between antioxidants and cancer prevalence. (Pharmacol Biochem Behav. 2006 Dec. 4; Expert Opin Investig Drugs. 2007 January; 16(1):25-32; COPD. 2004; 1(2):255-77; Review. Indian J Exp Biol. 2006 October; 44(10):816-20; Urol Res. 2005 December; 33(6):448-52; Oncol Rep. 2006 September; 16(3):617-24; Curr. Med. Chem. 2006; 13(1):87-98. Review; J Phys Chem B Condens Matter Surf Interfaces Biophys. 2005 Dec. 22; 109(50):24197-202).

The present invention provides an edible preparation for the prevention and/or activation of defense mechanisms in the human body against conditions, disorders or diseases relating to environmental pollution, namely smoking, these benefits are provided by the antioxidant effects of phytonutrients (flavonoids, carotenoids, lutein, terpenes, anthocyanins, sulfuranes, among others,) employed in the preparation thereof that act as potent inhibitors of carcinogenic cells and free radicals, as well as protective agents of cellular DNA. The main components of the invention are Aloe barbadensis, leaf extract, resveratrol, lemon peel extract, orange peel extract and pharmaceutically acceptable excipients. However, it should be noted that the preparation might optionally include other citric antioxidants, vitamins, minerals, electrolytes, energy and dietary supplements.

Resveratrol (3,5,4′-trihydroxystilbene) has been identified as a constituent found not only in the grape skin (Soleas et al. (1995) Am. J. Enol. Vitic. 46(3):346-352) but also in peanut, eucalyptus and other plant species. Golberg et al. (1995), Am. J. Enol. Vitic. 46(2):159-165. There has been a great deal of interest in the fungicidal activity of the compound and its correlation with fungal infection resistance. Id. on 159. Resveratrol is available in the market (usually as the trans isomer, e.g. from Sigma Chemical Company, St. Louis, Mo.) or may be isolated from the wine of the skin of the grape, or it may be chemically synthesized. Usually the synthesis is performed by a Witting reaction joining two substituted phenols through a double styrene bond, as described in Moreno-Manas et al. (1985) Anal. Chem. 81:157-61 and modified by other authors (Jeandet et al. (1991) Am. J. Enol. Vitic. 42:41-46; Goldberg et al. (1994) Anal. Chem. 66:3959-63).

There are more studies on the trans-resveratrol than on the cis isomer thereof; however, the cis isomer seems to be equally important from a biological viewpoint. Numerous utilities of resveratrol isomers have been proposed and evaluated. In Jang et al. (1997) Science 275:218-220 it is demonstrated that the resveratrol has a chemopreventive activity against cancer in assays featuring three main stages of the carcinogenesis. That is, the authors observed that the compound: (1) acted as antioxidant and antimutagenic and induced phase II enzymes which metabolize the drug (“anti-initiation” activity); (2) produced anti-inflammatory effects and inhibited the cyclo-oxygenase and hydroperoxidase (“anti-promoting” activity), and (3) induced promyelocytic differentiation of leukemic cells (“anti-promoting” activity). Furthermore, resveratrol has been extensively studied for its correlation with the cardiovascular utility of red wine. See, e.g., Bertelli et al., supra; Pace-Asciak et al. (1995), Clinica Chimica Acta 235:207-2191; and Frankel et al. (Apr. 24, 1993), The Lancet 341:1104. Neurological uses have also been proposed (Lee et al. (1994), Society for Neuroscience Abstracts 20(1-2): 1648).

The Aloe genus comprises approximately 600 species of plants. (Capasso et al, “Aloe and Its Therapeutic Use”, Phytotherapy Research, 1998; 12:124-127). One of these species is known as Aloe Barbadensis Miller and is found in Central America where it is usually called Aloe Vera. The Aloe is comprised mainly of the mucilagenous tissue of parenchyma, which is usually extracted by incision from the fresh leaves of the plants, and is immediately used as the juice or gel obtained therefrom or dried (e.g. by lyophilization) until the time of use. The aloe contains mainly glycoproteins, saccharides and enzymes. The polysaccharides are mainly partly acetylated glucomannans differing in their glucose, mannose ratio, their level of acetylation, the polysaccharide linearity or branching and their molecular weight. Other saccharides found in Aloe include glucose, mannose, L-rhamnose and aldopentose. The enzymes found in Aloe include oxidase, amylase, catalase, lipase and alkaline phosphatase. Other components found in Aloe include cholesterol, triglycerides, steroids, sitosterol, lignins, uric acid, gibberellin, lecithin-type substances and salicylic acid. (Shelton, “Aloe Vera: Its Chemical and Therapeutic Properties”, International Journal of Dermatology, 1991; 30(10):679-683).

### SUMMARY OF THE INVENTION

In a first embodiment, the invention provides a method for the prevention and/or activation of defense mechanisms in the human body against conditions, disorders or diseases relating to the presence of free radicals, such as; environmental pollution, aging or smoking.

In a second embodiment, the invention provides a phytonutrient-and-citric-based edible preparation for the prevention and/or activation of defense mechanisms in the human body against conditions, disorders or diseases relating to the presence of free radicals, such as environmental conditions or smoking use, benefits that are provided by the antioxidant effects of the components thereof.

In a third embodiment of the invention, an edible preparation is provided in liquid form (syrup, solution) made from phytonutrients and citrics for the prevention and/or activation of defense mechanisms in the human body against conditions, disorders or diseases relating to the presence of free radicals, such as environmental pollution or smoking.

In a fourth embodiment of the invention, a phytonutrient-and-citric-based edible preparation is provided in solid form (tablets, capsules, pills) for the prevention and/or activation of defense mechanisms in the human body against conditions, disorders or diseases relating to the presence of free radicals, such as environmental pollution or smoking.

In a fifth embodiment of the invention, a phytonutrient-and-citric-based chewing gum is provided for the prevention and/or activation of defense mechanisms in the human body against conditions, disorders or diseases relating to the presence of free radicals, such as environmental pollution or smoking.

In a sixth embodiment of the invention, a kit or pack is provided that contains an edible dosage form of the invention and tobacco in any of its commercial presentations for consumption.

### DETAILED DESCRIPTION OF THE INVENTION

Within the scope of the description of the invention, the terms employed hereinabove and hereinafter are defined as follows:

The expression “method for the prevention and/or action of defense mechanisms in the human body” refers to a therapeutic method comprising the administration to a mammal, namely a human being, of an effective amount of a phytonutrient-and-citric-based edible dosage, during or after tobacco consumption and which is intended to prevent pathologies, conditions, disorders or diseases relating to the presence of free radicals in the human body. Likewise, the therapeutic method of the invention enables activation of defense mechanisms in the human body to reduce conditions, disorders or diseases relating to the presence of free radicals in the body.

The expression “edible preparation” refers to all those preparations and/or compositions that may be consumed by anybody at any time of the day and include, without limitation solid and liquid forms.

The expression “dosage form” refers to any solid or liquid form under which the preparation of the invention may be presented and includes, but not limited to, tablets, capsules, pills, candies, chewing gums, powder, syrup, solutions, suspensions, toothpaste, mouth wash, cookies, biscuits, foodstuffs or beverages.

The expression “extract” refers to the resulting product from the intimate contact between a solvent and the respective vegetal material (leaves, peel, etc.) and include all pharmaceutically acceptable polar and apolar extracts, preferably the extracts employed in the preparation of the invention correspond to extracts in water, alcohol or hydroalcohol. Likewise, this extract may be subject to customary processes of solvent elimination by rotoevaporation or other processes contained in the state of the art for obtaining a solid.

The expressions “orange peel extract” and “lemon peel extract” refer to extracts derived from the peels of the fruits citrus sinensis and citrus limon, respectively, and include the extract from any of the varieties of these plants.

The expression “other citric antioxidants” refers to any isolated molecule, drug or extract having antioxidant effects that may be obtained from any of the 145 species of the citrus genus.

The expression “pharmaceutically acceptable excipients” should be understood as all those conventional excipients employed in the formulation of preparations and include bonding agents, charging stock, lubricants for tablet compression, disintegrators, moisturizing agents, suspension agents, emulsifying agents, non-aqueous vehicles, base gum, preservatives, sweeteners, flavorings and colorants.

The expression “kit or pack” refers to a container comprising the preparation of the invention in any of its dosage forms and a commercial presentation form of the tobacco. The container may be manufactured from any material suitable to the characteristics of the dosage form, in any conventional shape, such as paper box, cardboard box, plastic bottle, glass bottle, resealable bag, etc.

The expression “commercial presentation form of tobacco” refers to all forms in which tobacco is sold, e.g. cigarettes, pipes, etc. and includes any marketing unit; packs, half packs, unit packaging, etc.

The present invention teaches a method for the prevention and/or activation of defense mechanisms in the human body against conditions, disorders or diseases relating to the presence of free radicals, such as environmental pollution or smoking, which comprises the administration of phytonutrient-and-citric-based edible preparations in the form of tablets, capsules, pills, candies, gums, chewing gums, powder, syrup, liquid, cookie, biscuit, foodstuff or beverage or other edible forms, and which preparations comprise: Aloe barbadensis leaf extract, resveratrol, lemon peel extract, orange peel extract, pharmaceutically acceptable excipients and optionally other citric antioxidants, vitamins, minerals, electrolytes, energy and dietary supplements.

The preferred dosage forms of the invention are liquid forms of the solution, syrup or suspension type; the solid forms of the tablet or capsule and chewing gum type, which may be consumed by subjects before, during or after tobacco consumption. This will ensure the creation of a habit of association between the use of the preparation and the habit of smoking.

Preferably the preparation contains a combination of phytonutrients and citrics that provide natural antioxidants for the prevention and/or activation of defense mechanisms in the human body against conditions, disorders or diseases relating to the presence of free radicals such as environmental pollution or smoking. Within the broad range of phytonutrients and citrics, the present invention teaches a preparation that provides natural antioxidants in the form of an edible dosage form containing: 20-40% Aloe barbadensis leaf extract, 20-40% resveratrol, 20-40% lemon peel extract, 20-40% orange peel extract and pharmaceutically acceptable exicipients for conditioning the several forms of preferred dosage of the invention. Likewise, the preparations of the invention may optionally include other citric antioxidants, vitamins, minerals, electrolytes, energy and dietary supplements, if the nutritional or energy value of the composition is to be increased.

Likewise, the pharmaceutically acceptable excipients employed in the formulation of the present invention correspond to all those excipients necessary for adapting the edible dosage forms, bonding agents, charging stock, lubricants for tablet compression, disintegrators, moisturizing agents, suspension agents, emulsifying agents, non-aqueous vehicles, base gum, preservatives, sweeteners, flavorings and colorants are included in this group.

Solid non-toxic conventional vehicles for solid forms include, for example, pharmaceutical grades of manitol, lactose, starch, magnesium stearate, sodium saccharine, talc, cellulose, glucose, sucrose, magnesium carbonate and the like. The liquid compositions may be prepared, for example, by dissolving, dispersing, etc. one or more extracts and optional pharmaceutical adjuvants in a solvent, such as, for example, water, saline solution, aqueous dextrose, glycerol, ethanol and the like, in order to form thus a solution or suspension. If so desired, the preparation to be administered may also contain small amounts of non-toxic auxiliary substances such as moisturizing agents or emulsifiers, pH buffering agents and the like, for example sodium acetate, sorbitan monolaurate, triethanolamine sodium acetate, triethanolamine oleate, etc. The process of preparing such forms of dosage are known, or will be obvious for a person conversant with the art, see for example: “Remington's Pharmaceutical Sciences”.

The following examples intend to illustrate some of the preferred embodiments of the invention but not to limit in any way the scope of the described invention:

### EXAMPLE 1

Chewing gum for the prevention and/or activation of defense mechanisms in the body against conditions, disorders or diseases relating to environmental pollution, namely smoking.

This preparation is formulated in a chewing gum base for consumption before and/or during and/or after tobacco consumption for providing a mixture of antioxidants that act as a potent inhibitor of free radicals and other toxics, precluding their harmful effects in the human body. The manufacture of chewing gum and the use of the gum base in such manufacture is well known for technicians conversant with the art and may be found in U.S. Pat. Nos. 4,238,475 (Witzel et al) and 4,671,961 (Mansukth et al) and “Sugar confectionery and chocolate manufacture” (R. Lees y E. B. Jackson, 1992). The active ingredients are expressed in a w/w percentage unless otherwise stated. A unit represents a strip, tablet or ball of chewing gum.

8% Aloe barbadensis leaf extract

### 8% Resveratrol

8% Lemon peel extract
8% Orange peel extract
6% Gum base

### 60% Sugar

### 10% Glucose

### 1.2% Flavoring

### 0.5% Emulsifier

### EXAMPLE 2

Tablets for the for the prevention and/or activation of defense mechanisms in the human body against conditions, disorders or diseases relating to environmental pollution namely, smoking.

These dosage forms may be prepared by traditional methods of mixing, filling or compressing tablets. Repeated mixing operations may be employed to distribute antioxidant agents in all preparations using considerable amounts of charging stock. Said operations are obviously customary in the state of the art. The tablets may be coated according to the methods fully known in the normal practice.

| Ingredient | Amount (mg per tablet) | | |
|---|---|---|---|
| Aloe barbadensis leaf extract | 10 | 50 | 100 |
| Resveratrol | 10 | 50 | 100 |
| Lemon peel extract | 10 | 50 | 100 |
| Orange peel extract | 10 | 50 | 100 |
| Extracto de cáscara de naranja | | | |
| Starch | 6.2 | 6.0 | 14.0 |
| Microcrystalline Cellulose | 24.8 | 22 | 41.5 |
| Monohydrate Lactose | 78.25 | 70.5 | 142.5 |
| Magnesium Stearate | 0.75 | 1.5 | 3.0 |
| Total weight of tablet core | 150 | 300 | 600 |

### EXAMPLE 3

Liquid preparations for the for the prevention and/or activation of defense mechanisms in the human body conditions, disorders or diseases relating to environmental pollution, namely smoking.

The liquid preparations administered via oral may be in the form, for example, of solutions, syrups, suspensions or elixirs, or may be presented as a dried product for its reconstitution with water, or other suitable vehicle, before use.

Solution

| | |
|---|---|
| Aloe barbadensis leaf extract | 5% |
| Resveratrol | 5% |
| Lemon Peel Extract | 5% |
| Orange Peel Extract | 5% |
| Sweeteners | 2% |
| Preservatives | 0.7% |
| Water | c.s.p. 100 ml |

1. A method for the prevention and/or activation of defense mechanisms in the human body against conditions, disorders or diseases relating to the presence of free radicals, such as environmental pollution or smoking, comprising the administration of an effective amount of a phytonutrient-and-citric-based edible dosage characterized in that it comprises: Aloe barbadensis leaf extract, resveratrol, lemon peel extract, orange peel extract, pharmaceutically acceptable excipients and optionally other citric antioxidants, vitamins, minerals, electrolytes, energy and dietary supplements.

2. A method for the prevention and/or activation of defense mechanisms in the human body against conditions, disorders or diseases relating to the presence of free radicals, such as environmental pollution or smoking, comprising the administration of an effective amount of a phytonutrient-and-citric-based edible dosage characterized in that it comprises: i. 20-40% Aloe barbadensis leaf extract; ii. 20-40% Resveratrol; iii. 20-40% Lemon peel extract; iv. 20-40%, Orange peel extract; v. Pharmaceutically acceptable excipients and vi. optionally other citric antioxidants, vitamins, minerals, electrolytes, energy and dietary supplements.

3. A method according to claims 1 and 2 wherein the dosage form is selected from tablet, capsule, pill, candy, gum, chewing gum, syrup, liquid, cookie, biscuit, foodstuff or beverage.

4. A method according to claims 1 and 2 wherein the dosage form is consumed before and/or during and/or after tobacco consumption.

5-15. (canceled)
